(19) United States
(12) Patent Application Publication
(10) Pub. No.: US 2008/0234326 A1
(43) Pub. Date: Sep. 25, 2008

(54) Novel Salts of Pantoprazole and (S) - Pantoprazole

(75) Inventors: Rolf-Peter Hummel, Radolfzell (DE); Bernhard Kohl, Konstanz (DE); Bernd Mueller, Konstanz (DE); Ernst Sturm, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/586,753

(22) PCT Filed: Jan. 27, 2005
(86) PCT No.: PCT/EP05/50334

§ 371 Date: Jul. 21, 2006

(30) Foreign Application Priority Data
Jan. 28, 2004 (EP) 04001775.8

(51) Int. Cl. A61K 31/4439; C07D 401/12; A61P 1/04

(52) U.S. Cl. 514/338; 546/273.7

(57) ABSTRACT

The invention relates to pantoprazole and (S)-pantoprazole salts and to medicaments comprising these compounds.

### SUBJECT-MATTER OF THE INVENTION

The present invention relates to novel salts of the active compound pantoprazole. The novel salts can be used in the pharmaceutical industry for preparing medicaments.

### BACKGROUND OF THE INVENTION

Owing to their H⁺/K⁺-ATPase-Inhibitory action, pyridin-2-ylmethylsulphinyl-1H-benzimidazoles, such as those known, for example, from EP-A-0005129, EP-A-0166287, EP-A-0174726 and EP-A-0268956 are of considerable importance in the therapy of disorders associated with an increased secretion of gastric acid.

Examples of active compounds from this group which are commercially available or in clinical development are 5-methoxy-2-[(4-methoxy-3,5-dimethyl-2-pyridinyl)methylsulphinyl]-1H-benzimidazole (INN: omeprazole), (S)-5-methoxy-2-[(4-methoxy-3,5-dimethyl-2-pyridinyl)methylsulphinyl]-1H-benzimidazole (INN: esomeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl]-1H-benzimidazole (INN: lansoprazole), 2-[(3-methoxypropoxy)-3-methylpyridin-2-yl]methylsulphinyl]-1H-benzimidazole (INN: rabeprazole) and 5-methoxy-2-((4-methoxy-3,5-dimethyl-2-pyridylmethyl)sulphinyl)-1H-imidazo[4,5-b]pyridine (INN: tenatoprazole).

The above mentioned sulphinyl derivatives which, owing to their mechanism of action, are also referred to as proton pump inhibitors or, abbreviated, as PPI, are chiral compounds.

### DESCRIPTION OF THE RELATED ART

For the first time, the international patent application WO92/08716 describes a chemical process, which allows pyridin-2-ylmethylsulphinyl-1H-benzimidazoles to be separated into their optical antipodes. The compounds mentioned as being prepared in an exemplary manner include, inter alia, the compounds (+)- and (−)-difluoromethoxy-2-[(3,4-dimethoxy-2-pyridinyl)methylsulphinyl]-1H-benzimidazole [=(+)- and (−)-pantoprazole]. The International patent application WO92/08716 mentions that the optical antipodes of the pyridin-2-ylmethylsulphinyl-1H-benzimidazoles, i.e. the (+)- and (−)-enantiomers or the (R)- and (S)-enantiomers, are useful as active compounds in medicaments for the treatment of gastrointestinal disorders. For the mode of application and the dosage of the active compounds, reference is made, inter alia, to the European patent 166 287.

The International patent applications WO94/24867 and WO94/25028 claim the use of the compounds (+)- and (+)-pantoprazole for treating gastric disorders in humans. Each stereoisomer is said to have medical advantages compared to the respective other stereoisomer. The descriptions also mention a number of different possible salts of the stereoisomers, and particular preference is given to the sodium salt.

In international patent application WO94/27988, certain salts of (+) and (−)-omeprazole and methods for their preparation are disclosed.

The international patent application WO97/41114 describes a certain process for preparing magnesium salts of pyridin-2-ylmethylsulphinyl-1H-benzimidazoles. What is described in an exemplary manner is, inter alia, the preparation of the magnesium salt of racemic pantoprazole. According to the given analytical data, the salt that is prepared is racemic pantoprazole magnesium in anhydrous form.

The international patent application WO00/10995 describes the dihydrate of the magnesium salt of racemic pantoprazole.

The international patent application WO99/27917 relates to a peroral medicament preparation in the form of a pellet or a tablet for acid-labile pyridine-2-ylmethylsulfinyl-1H-benzimidazoles comprising an alkaline pellet or tablet core and a coating made of one or more film formers which can be utilized for gastric juice resistant coatings, whereby the coating which is in direct contact with the pellet or tablet core is comprised of a neutralized film former.

The international patent application WO02/45686 relates to the field of pharmaceutical technology and describes a pharmaceutical preparation in the form of a paste comprising an acid-labile active ingredient, in particular an acid-labile proton pump inhibitor, such as pantoprazole.

The international patent application WO 2004/013126 relates to (−)-pantoprazole magnesium and its hydrates and to medicaments comprising these compounds.

A common property of all of the abovementioned PPI is their sensitivity to acids (ultimately essential for effectiveness) which becomes apparent in their strong tendency to decompose in a neutral and in particular an acidic environment, giving rise to intensely coloured decomposition products. In the past, there has been no lack of considerable efforts, in spite of the sensitivity of the PPI to adds, to obtain stable and storable oral dosage forms comprising these PPI. Such stable and storable oral dosage forms (for example tablets or capsules) are now obtainable. However, the preparation of these oral dosage forms is relatively complicated, and with respect to the packaging too, certain complicated precautions have to be taken so that the dosage forms are sufficiently stable on storage even under extreme storage conditions (for example in tropical regions at high temperatures and high atmospheric humidity). Furthermore, in the past, there has been no lack of efforts to tailor the release of the PPI in the human body in the best possible manner to the respective requirements.

### DESCRIPTION OF THE INVENTION

It has now been found that the sodium salt of (−)- or (S)-pantoprazole, which is particularly preferred in the International patent application WO 94/24867, does not form a stable storage form. During various attempts to obtain stable oral dosage forms for pantoprazole and (S)-pantoprazole, it has now been found that certain salts, which have not been expressly described in the prior art, have unexpected and advantageous properties, either with regard to stability characteristics, and/or with regard to their pharmacodynamic and/or pharmacokinetic properties. On account of these properties, these new salts are expected to be useful as therapeutics in human medicine.

Accordingly, the invention provides in a first aspect the calcium, potassium, zinc and aluminium salts of pantoprazole and (S)-pantoprazole [=(−)-pantoprazole]. Preferably, the invention provides these salts in the form of their stable hydrates.

Expressly, the invention provides the compounds
calcium (S)-bis{[5-difluoromethoxy)]-2-[(3,4-dimethoxy-2-pyridinyl)methylsulphinyl]-1H-benzimidazolide},
zinc (S)-bis{[5-(difluoromethoxy)]-2-[(3,4-dimethoxy-2-pyridinyl)methylsulphinyl]-1H-benzimidazolide},
aluminium (S)-tris{[5-(difluoromethoxy)]-2-[(3,4-dimethoxy-2-pyridinyl)methylsulphinyl]-1H-benzimidazolide},
potassium (S){[5-difluoromethoxy)]-2-(3,4-dimethoxy-2-pyridinyl)methylsulphinyl]-1H-benzimidazolide},
calcium bis{[5-(difluoromethoxy)]-2-[(3,4-dimethoxy-2-pyridinyl)methylsulphinyl]-1H-benzimidazolide},
zinc bis{[5-(difluoromethoxy)]-2-[(3,4-dimethoxy-2-pyridinyl)methylsulphinyl]-1H-benzimidazolide},
aluminium tris{5-(difluoromethoxy)]-2-[(3,4-dimethoxy-2-pyridinyl)methylsulphinyl]1H-benzimidazolide}
   and
potassium {[5-(difluoromethoxy)]-2-[(3,4-dimethoxy-2-pyridinyl)methylsulphinyl]-1H-benzimidazolide},
   and the hydrates of these compounds.

The salts according to the invention and their hydrates can be used for the treatment and prevention of all disorders, which can be treated or prevented by using PPI. In particular, the salts according to the invention and their hydrates can be used for treating gastric disorders.

The new salts of pantoprazole and (S)-pantoprazole are prepared in a manner known per se by reacting pantoprazole or (S)-pantoprazole with a suitable calcium, zinc, potassium or aluminium base, for example a calcium alkoxide, a potassium hydroxide etc., or from a readily soluble pantoprazole or (S)-pantoprazole salt (for example pantoprazole or (S)-pantoprazole sodium) using e.g. a zinc or aluminium salt in water or in mixtures of water with polar organic solvents (for example alcohols, preferably methanol, ethanol or isopropanol, or ketones, preferably acetone).

Salts suitable for use in the process are, for example, zinc chloride, calcium bromide, zinc fluoride, potassium iodide, aluminium formate, aluminium acetate, zinc propionate, calcium gluconate or potassium carbonate it is also possible to react alkoxides (for example aluminium methoxide, zinc ethoxide, potassium (iso)propoxide or calcium butoxide) in an alkoholate medium with pantoprazole, pantoprazole sodium, (S)-pantoprazole or (S)-pantoprazole sodium and to crystallise the obtained pantoprazole or (S)-pantoprazole salts, if desired in form of their hydrates by addition of water. Furthermore, it is possible to recrystaillse obtained hydrates from, e.g., methanol/water mixtures.

For use in solid, in particular oral, pharmaceutical formulations, the salts according to the invention are milled in order to obtain crystals with a particle size distribution of 90%, preferably 99% below 100 μm.

According to the invention, “(S)-pantoprazole” is understood to include “(S)-pantoprazole, substantially free of the (R)-enantiomer.” “Substantially free” in this context means that (S)-pantoprazole contains less than 10% by weight of (S)-pantoprazole. Preferably, “substantially free” means that (S)-pantoprazole contains less than 5% by weight of (R)-pantoprazole. In the most preferred embodiment, “substantially free” means that (S)-pantoprazole contains less than 1% by weight of (S)-pantoprazole.

### EXAMPLES

### 1. Pantoprazole-Zn-Salt

60 g (139 mmol) of Pantoprazole-Na sesquihydrate are added to 1 l of water and dissolved. 11.37 g (83 mmol) of zinc chloride are dissolved in 200 mlof water. The moody solution of zinc chloride is filtered before use and added to the solution of Pantoprazole-Na sesquihydrate at room temperature within 30 minutes. The suspension is stirred for one additional hour and the precipitation is filtered. The salt is washed with 500 ml of water free of chloride, dried at 60° C. in vacuum and yields 95% of theory.

Mp: 166° C. (degradation), water content (kari-Fischer) 2.1%.

### CHN-Analysis:

| | expected | |
|---|---|---|
| | (monohydrate) | found |
| C | 45.32 | 45.08 |
| H | 3.57 | 3.66 |
| N | 9.91 | 9.88 |

### 2. Pantoprazole-Ca-Salt

43.2 g (100 mmol) of Pantoprazole-Na sesquihydrate are added to 600 ml of water and dissolved. 8.1 g (55 mmol) of calcium chloride are dissolved in 50 ml of water. The solution of calcium chloride is added to the solution of Pantoprazole-Na sesquihydrate at room temperature within 5 minutes. The suspension is stirred for additional 1.5 hours and the precipitation is filtered. The salt is washed with 300 ml of water, dried at 40-45° C. in vacuum and yields 73% of theory. Mp: 158.5° C. (degradation), water content (Karl-Fischer) 7.5%.

### CHN-Analysis:

| | expected | |
|---|---|---|
| | (dihydrate) | found |
| C | 44.75 | 44.25 |
| H | 3.99 | 4.09 |
| N | 9.79 | 9.67 |

### 3. Pantoprazole-1-Salt

2.0 g (5.2 mmol) of Pantoprazole are dissolved in 10 ml of aceton. The solution is heated to 50° C. After 0.22 g (5.2 mmol) lithium hydroxide are added, the mixture is stirred at this temperature for 1.5 hours. For removal of water the solution is coevaporated once with isopropanol. Solvents are removed in the evaporator to obtain the product Mp: 78-80° C. (degradation), water content (Karl-Fischer) 8.3%.

### CHN-Analysis:

| | expected | |
|---|---|---|
| | (dihydrate) | found |
| C | 45.18 | 45.50 |
| H | 4.27 | 4.30 |
| N | 9.88 | 9.80 |

### 4. (−)-Pantoprazole-Zn-Salt

1.0 g (2.3 mmol) of (−)-Pantoprazole-Na sesquihydrate is added to 10 ml of water and dissolved. 0.19 g (1.4 mmol) of zinc chloride is dissolved in 2 ml of water and is added to the solution of (−)-Pantoprazole-Na sesquihydrate at room temperature within 5 minutes. The suspension is stirred for additional 2 hours and the precipitation is filtered. The salt is washed with 10 ml of water free of chloride, dried at 60° C. in vacuum and yields 84% of theory. Mp: 172° C. (degradation), water content (Karl-Fischer) 2.4%.

### CHN-Analysis:

| | expected | |
|---|---|---|
| | (monohydrate) | found |
| C | 45.32 | 44.92 |
| H | 3.57 | 3.72 |
| N | 9.91 | 9.82 |

### 5. (−)-Pantoprazole-Salt

12.2 g (28 mmol) of (−)-Pantoprazole-Na sesquihydrate are added to 160 ml of water and dissolved. 1.8 g (16 mmol) of calcium chloride are dissolved in 20 ml of water and are added to the solution of (+Pantoprazol-Na sesquihydrate at room temperature within 30 minutes. The suspension is stirred for additional 2 hours and the precipitation is filtered. The salt is washed with 100 ml of water free of chloride, dried at 60° C. in vacuum and yields 70% of theory. Mp: 158.5° C. (degradation), water content (Karl-Fischer) 6.5%.

### CHN-Analysis:

| | expected | |
|---|---|---|
| | (dihydrate) | found |
| C | 44.75 | 45.02 |
| H | 3.99 | 4.08 |
| N | 9.79 | 9.81 |

### 6. (−)-Pantoprazole-K-Salt

10 g (26 mmol) of (−)-Pantoprazole are added to 25 ml of water. 3.8 g (16 mmol) of potassium hydroxide solution (w/w=30%) are added to the suspension at room temperature and heated to 40-45° C. to get a solution. The suspension is stirred for 30 minutes and cooled down to room temperature. Product crystals are added to start the crystallisation and the mixture is cooled down to 0° C. and stirred for several hours. The precipitation is filtered off. The product is dried at 40° C. in vacuum and yields 11% of theory. Mp: 83.8° C. (degradation), water content (Karl-Fischer) 10.0%.

### CHN-Analysis:

| | expected | |
|---|---|---|
| | (dihydrate) | found |
| C | 42.01 | 42.42 |
| H | 3.97 | 4.19 |
| N | 9.18 | 9.25 |

### Commercial Utility

The pantoprazole and (S)-pantoprazole salts and their hydrates have useful pharmacological properties, rendering them commercially utilizable. In particular, they have a pronounced inhibitory effect on the secretion of gastric add and excellent gastrointestinal protective action in warm-blooded animals, in particular man. Here, the compounds according to the invention are distinguished by a highly selective action, an advantageous duration of action, a particularity high bioavailability, a metabolisation profile that is uniform among different individuals, the lack of significant side-effects and a wide therapeutic spectrum.

In this context, “gastrointestinal protection” is to be understood as the prevention and treatment of gastrointestinal disorders, in particular gastrointestinal inflammatory disorders and lesions (such as, for example, Ulcus ventriculi, Ulcus duodeni, gastrtis, irritable bowel owing to an increased production of acid or as a result of medicaments, GERD, Crohn's disease, IBD) which may be caused, for example, by microorganisms (for example Helicobacter pylori), bacterial toxins, medicaments (for example certain antiphlogistics and antirheumatic drugs), chemicals (for example ethanol), gastric acid or stress.

With their excellent properties, the pantoprazole and (S)-pantoprazole salts and their hydrates are, in various models for the determination of antiulcerogenic and antisecretory properties, surprisingly different to prior art compounds, in particular with respect to their stability and their metabolization properties and with regard to their pharmacodynamic and pharmacokinetic characteristics and with regard to their bioavailability profile. Owing to these properties, the pantoprazole and (S)-pantoprazole salts and their hydrates seem to be highly suitable for use in human and veterinary medicine, where they are used, in particular, for the treatment and/or prophylaxis of gastrointestinal disorders.

Accordingly, the invention furthermore provides the use of the pantoprazole and (S)-pantoprazole salts according to the invention and of their hydrates for the treatment and/or prophylaxis of the abovementioned diseases.

The invention also embraces the use of the pantoprazole and (S)-pantoprazole salts according to the invention and of their hydrates for preparing medicaments used for the treatment and/or prophylaxis of the abovementioned diseases.

The invention also provides medicaments comprising the pantoprazole and (S)-pantoprazole salts according to the invention and/or their hydrates.

The medicaments are prepared by processes known per se which are familiar to the person skilled in the art. As medicaments, the pantoprazole and (S)-pantoprazole salts according to the invention and their hydrates are employed either as such or, preferably, in combination with suitable pharmaceutical auxiliaries or carriers in the form of tablets, coated tablets, capsules, suppositories, plasters (for example as TTS), emulsions, suspensions or solutions, where the content of active compound is advantageously from about 0.1 to about 95% and where it is possible to produce pharmaceutical dosage forms (for example flow-release forms or enteric forms) which, by the appropriate choice of auxiliaries and carriers, are tailored for the active compound and/or the desired onset of action and/or the duration of action.

The auxiliaries or carriers suitable for the desired pharmaceutical formulations are known to the person skilled in the art. In addition to solvents, gel formers, suppository bases, tabletting auxiliaries and other carriers for active compounds, it is possible to use, for example, antioxidants, dispersants, emulsifiers, antifoams, flavour-masking agents, preservatives, solubilizers, colorants or, in particular, permeation promoters and complex formers (for example cyclodextrins).

The pantoprazole and (S)-pantoprazole salts according to the invention and their hydrates can be administered orally, parenterally or percutaneously.

In human medicine, it has generally been found to be advantageous to administer the pantoprazole and (S)-pantoprazole salts according to the invention and their hydrates, when given orally, in a daily dose of from about 0.1 to about 2, preferably about 0.2 to about 1.5 and in particular about 0.3 to about 1.1, mg/kg of body weight [based on pantoprazole or (S)-pantoprazole, respectively], if appropriate in the form of a plurality of, preferably 1 to 4, individual doses, to obtain the desired result. For parenteral treatment, it is possible to use similar or (in particular when the active compounds are administered intravenously) generally lower dosages. The optimum dosage and the type of administration of the active compounds required in each case can easily be determined by the person skilled in the art.

A further aspect of the invention is thus a medicament, comprising a pantoprazole or (S)-pantoprazole salt according to the invention and/or its hydrate(s) together with customary auxiliaries, where the single dose comprises from about 10 to about 100 mg of pantoprazole or (S)-pantoprazole, respectively.

A further aspect of the Invention is a medicament, comprising a pantoprazole or (S)-pantoprazole salt according to the invention and/or its hydrate(s) together with customary auxiliaries, where the single dose comprises from about 20 to about 80 mg of pantoprazole or (S)-pantoprazole, respectively.

A further aspect of the invention ^{_{the use of a pantoprazole or (S)-pantoprazole salt according to the invention and/or its hydrate(s) for treating gastrointestinal disorders.}}

A further aspect of the Invention is the use of a (S)-pantoprazole salt according to the Invention and/or its hydrate(s) for treating gastrointestinal disorders in patients who are slow metabolizers.

A further aspect of the Invention is the use of a pantoprazole or (S)-pantoprazole salt according to the invention and/or its hydrate(s) for treating gastrointestinal disorders in patients who have a risk of drug interactions.

A further aspect of the invention is the use of a pantoprazole or (S)-pantoprazole salt according to the invention and/or its hydrate(s) for treating gastrointestinal disorders in patients who need an Inhibition of acid secretion for an extended period of time.

A further aspect of the invention is a medicament for treating gastrointestinal disorders for use in patients who are slow metabolizers, comprising a (S)-pantoprazole salt according to the Invention and/or its hydrate(s) together with customary auxiliaries, where the single dose comprises from about 10 to about 100 mg of (S)-pantoprazole.

A further aspect of the invention is a medicament for treating gastrointestinal disorders for use in patients who are slow metabolizers, comprising a (S)-pantoprazole salt according to the invention and/or its hydrate(s) together with customary auxiliaries, where the single dose comprises from about 20 to about 80 mg of (S)-pantoprazole.

A further aspect of the invention is a medicament for treating gastrointestinal disorders for use in patients who have a risk of drug interactions, comprising a pantoprazole or (S)-pantoprazole salt according to the invention and/or its hydrate(s) together with customary auxiliaries, where the single dose comprises from about 10 to about 100 mg of pantoprazole or (S)-pantoprazole, respectively.

A further aspect of the invention is a medicament for treating gastrointestinal disorders for use in patients who have a risk of drug interactions, comprising a pantoprazole or (S)-pantoprazole salt according to the invention and/or its hydrate(s) together with customary auxiliaries, where the single dose comprises from about 20 to about 80 mg of pantoprazole or (S)-pantoprazole, respectively.

A further aspect of the invention is a medicament for treating gastrointestinal disorders for use in patients who need an inhibition of acid secretion for an extended period of time, comprising a pantoprazole or (S)-pantoprazole salt according to the invention and/or its hydrate(s) together with customary auxiliaries, where the single dose comprises from about 10 to about 100 mg of pantoprazole or (S)-pantoprazole, respectively.

A further aspect of the invention is a medicament for treating gastrointestinal disorders for use in patients who need an inhibition of acid secretion for an extended period of time, comprising a pantoprazole or (S)-pantoprazole salt according to the invention and/or its hydrate(s) together with customary auxiliaries, where the single dose comprises from about 20 to about 80 mg of pantoprazole or (S)-pantoprazole, respectively.

If pantoprazole or (S)-pantoprazole salts according to the invention and/or hydrates thereof are to be used for treating the abovementioned diseases, the pharmaceutical preparations may also comprise one or more pharmacologically active ingredients from other groups of medicaments. Examples that may be mentioned include tranquilizers (for example from the group of the benzodiazepines, e.g., diazepam), spasmolytic drugs (e.g., bietamiverine or camylofine), anticholinergic drugs (e.g., oxyphencyclimine or phencarbamide), local anesthetics (e.g., tetracaine or procaine), and optionally also enzymes, vitamins or amino acids.

In this context, particular emphasis is given to the combination of the compounds according to the invention with other pharmaceuticals which buffer or neutralize gastric acid or which inhibit the secretion of acid, such as, for example, antacids (such as, for example, magaldrate) or H₂ blockers (e.g., cimetidine, ranitidine), and with gastrin antagonists with the aim to enhance the main action in an additive or superadditive sense and/or to eliminate or reduce side-effects or to obtain a more rapid onset of action. Mention may also be made of the fixed or free combination with NSAIDs (such as, for example, etofenamate, diclofenac, indometacin, ibuprofen or piroxicam) for preventing the gastrointestinal damage caused by the NSAIDs, or with compounds, which modify gastrointestinal motility, or with compounds, which reduce the incidence of transient lower esophageal sphincter relaxation (TLOSR), or with antibacterial substances (such as, for example, cephalosporins, tetracyclins, penicillins, macrolides, nitroimidazoles or else bismuth salt) for controlling Helicobacter pylori. Antibacterial combination partners that may be mentioned include, for example, mezlocilin, ampicillin, amoxicillin, cefalothin, cefoxitin, cefotaxim, imipenem, gentamycin, amicacin, erythromycin, ciprofioxacin, metronidazole, carithromycin, azithromycin and combinations thereof (e.g., clarithro−mycin+metronidazole or amoxicillin+darithromycin).

1. A calcium, potassium, zinc, lithium or aluminium salt of pantoprazole or (S)-pantoprazole, or a hydrate thereof.

2. A calcium, potassium, zinc or aluminium salt of pantoprazole or (S)-pantoprazole, or a hydrate thereof.

3. A compound selected from the group consisting of calcium (S)-bis{[5-(difluoromethoxy)]-2-[(3,4-dimethoxy-2-pyridinyl)methyl-sulphinyl]-1H-benzimidazolide},
zinc (S)-bis{[5-(difluoromethoxy)]-2-[(3,4-dimethoxy-2-pyridinyl)methylsulphinyl]-1H-benzimidazolide},
aluminium (S)-tris{[5-(difluoromethoxy)]-2-[(3,4-dimethoxy-2-pyridinyl)methylsulphinyl]-1H-benzimidazolide},
potassium (S)-{[5-(difluoromethoxy)]-2-[(3,4-dimethoxy-2-pyridinyl)methylsulphinyl]-1H-benzimidazolide},
calcium bis{[5-(difluoromethoxy)]-2-[(3,4-dimethoxy-2-pyridinyl)methylsulphinyl]-1H-benzimidazolide},
zinc bis{[5-(difluoromethoxy)]-2-[(3,4-dimethoxy-2-pyridinyl)methylsulphinyl]-1H-benzimidazolide},
aluminium tris{[5-(difluoromethoxy)]-2-[(3,4-dimethoxy-2-pyridinyl)methylsulphinyl]-1H-benzimidazolide},
potassium {[5-(difluoromethoxy)]-2-[(3,4-dimethoxy-2-pyridinyl)methylsulphinyl]-1H-benzimidazolide},
and the hydrates of these compounds.

4. A compound selected from the group consisting of lithium (S)-{[5-(difluoromethoxy)]-2-[(3,4-dimethoxy-2-pyridinyl)methylsulphinyl]-1H-benzimidazolide},
lithium {[5-(difluoromethoxy)]-2-[(3,4-dimethoxy-2-pyridinyl)methylsulphinyl]-1H-benzimidazolide}, and the hydrates of these compounds.

5. A pantoprazole or (S)-pantoprazole salt according to claim 1, or a hydrate thereof, with a particle size distribution of 99% below 100 μm.

6. A pharmaceutical composition comprising a compound according to claim 1, together with a pharmaceutically acceptable auxiliary.

7. A pharmaceutical composition comprising a compound according to claim 1, together with a pharmaceutically acceptable auxiliary, where a single dose comprises from about 10 to about 100 mg of pantoprazole or (S)-pantoprazole.

8.-11. (canceled)

12. A method of treating a gastrointestinal disorder in a patient comprising administering to a patient in need thereof, a compound of claim 1 or a pharmaceutically acceptable salt thereof.

13. A method of treating a gastrointestinal disorder in a patient comprising administering to a patient in need thereof, a compound of claim 1 or a pharmaceutically acceptable salt thereof, wherein said patient is a slow metabolizer.

14. A method of treating a gastrointestinal disorder in a patient comprising administering to a patient in need thereof, a compound of claim 1 or a pharmaceutically acceptable salt thereof, wherein said patient has a risk of drug interactions.

15. A method of treating a gastrointestinal disorder in a patient comprising administering to a patient in need thereof, a compound of claim 1 or a pharmaceutically acceptable salt thereof, wherein said patient needs an inhibition of acid secretion for an extended period of time.

16. A pantoprazole or (S)-pantoprazole salt according to claim 2, or a hydrate thereof, with a particle size distribution of 99% below 100 μm.

17. A pantoprazole or (S)-pantoprazole salt according to claim 3, or a hydrate thereof, with a particle size distribution of 99% below 100 μm.

18. A pantoprazole or (S)-pantoprazole salt according to claim 4, or a hydrate thereof, with a particle size distribution of 99% below 100 μm.
